(19) United States
(12) Patent Application Publication
(10) Pub. No.: US 2005/0272817 A1
(43) Pub. Date: Dec. 8, 2005

(54) Dermatological use and a dermatological preparation

(76) Inventor: Pekka Heino, Helsinki (FI)

Correspondence Address: JACOBSON HOLMAN PLLC, 400 SEVENTH STREET N.W., SUITE 600, WASHINGTON, DC, 20004, US

(21) Appl. No.: 11/198,133

(22) Filed: Aug. 8, 2005

Related U.S. Application Data
(63) Continuation-in-part of application No. 10/239,882, filed on Oct. 7, 2002, now abandoned.

(30) Foreign Application Priority Data
Apr. 6, 2000 (FI) 20000818

(51) Int. Cl. A61K 31/198

(52) U.S. Cl. 514/567

(57) ABSTRACT

The present invention concerns a dermatological use, a dermatological preparation and a method of treatment to inhibit or reduce the thyroid hormone activity of L-thyroxine and its metabolite L-triiodothyronine, which accelerate cellular metabolism and proliferation. A topical pharmaceutical preparation containing a thyronine derivative inhibiting the thyroid hormone activity of L-thyroxine and L-triiodothyronine is used.

This application is a continuation-in-part application of U.S. application Ser. No. 10/239,882, filed Oct. 7, 2002, the disclosure of which is incorporated herein by reference, which is a nationalization of PCT/FI01/00100 filed Feb. 5, 2001 and published in English.

### FIELD OF INVENTION

This invention relates to a dermatological use, a dermatological preparation and a treatment with the same. In this invention it is treated of proliferative skin conditions, i.e. conditions manifested as accelerated multiplication of cells and as therewith associated disorders of cellular growth and differentiation.

### BACKGROUND OF THE INVENTION

The thyroid gland synthesizes and secretes thyroid hormones, which are iodine-containing derivatives of the amino acid thyronine. These hormones exhibit a biological activity known as thyroid hormone activity. The various compounds differ in their degree of thyroid hormone activity. Physiological and synthetic compounds possessing such activity are collectively known as thyroid hormone analogues. Hundreds of such compounds are known.

The most well-known physiological thyroid hormone analogue is L-thyroxine, or 3,3′,5,5′-tetraiodo-L-thyronine (T4), with four iodine atoms bound to the thyronine skeleton.

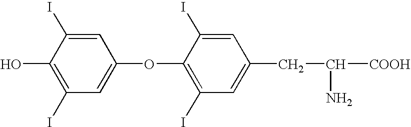

It is an essential human hormone regulating the general rate of metabolism, as well as activating cell proliferation (their multiplication, growth and differentiation). Abnormal increases in the amount of thyroxine, as in hyperthyroidism, cause acceleration of cellular and tissue metabolism, manifested clinically as thyroid hormone poisoning, or thyrotoxicosis. The symptoms of this hyper metabolic syndrome are also evident on the skin. In patients with psoriasis, for instance, the patient's disease typically worsens. T4 is mostly metabolized through de-iodination in the liver and peripheral tissues, including the skin, to L-triuodothyronine; 3,3′,5-triiodo-L-thyronine, T3, containing three iodine atoms.

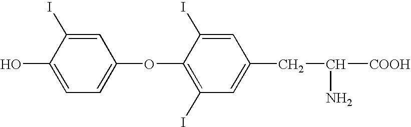

T3 has a thyroid hormone activity five times that of T4.

Some of the T4 is metabolized to 3,3′,5,5′-tetraiodothyropropionic acid, T4P, 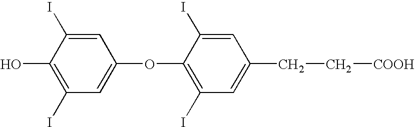
through deamination of the side chain of the thyronine skeleton or to 3,3′,5,5′-tetraiodothyroacetic acid, T4A, 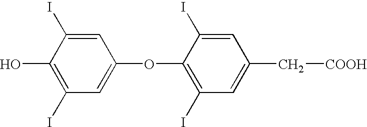
through side chain deamination and shortening. These tetraiodothyrocarboxylic acids possess only about one-fourth of the thyroid hormone activity of their parent compound, T4.

Patients with untreated hypothyroidism exhibit significant skin symptoms in addition to other organ symptoms related to the reduced metabolic rate caused by T4 (T3) deficiency. Acid glycosaminoglycans accumulate in the subdermal layer, and connective tissue fibers are reduced in quantity and changed in quality. The skin becomes cold, yellowish and dry. Its cornified outer layer, the epidermis, becomes thick and coarse. The elbows and knees especially may develop coarse, dirty brown hyperkeratosis, i.e. epidermal thickening, also known as the “dirty knee symptom”.

Systemic administration of T4 (or T3) quickly removes these symptoms and normalizes the skin.

Of the thyroid hormone analogues, only T4 and T3 are used in therapeutic practice, with one or the other being administered internally as hormone substitution therapy for hypothyroidism-D-Thyroxine, i.e. the D-isomer of thyroxine, which has substantially less thyroid hormone activity than T4, has been tested as an agent to reduce blood lipid levels (Farwell A P, Braverman L E (1996): Thyroid and Antithyroid Drugs. In Goodman & Gilman's The Pharmacological Basis of Therapeutics, 9th ed Eds. Hardman J G, Limbird L E, Molinoff P B, Rudden P W, Goodman Gilman A. McGraw-Hill, New York, p. 1383-1409).

In addition to internal administration, prior art also includes topical administration of thyroid hormone analogues (e.g. NZ 207923, U.S. Pat. Nos. 5,856,359, 5,951,989, WO 9640048). Topical administration has been undertaken to reduce the systemic side effects caused by the compounds' thyroid hormone activity and to find new indications for their use.

### SUMMARY OF THE PRESENT INVENTION

The present invention uses thyronine derivatives (e.g. T4A and T4P) possessing a weaker thyroid hormone activity than T4 to pharmacologically inhibit or reduce the thyroid hormone activity of T4 (and T3). When T4A and/or T4P, both of which have weak thyroid hormone activity compared with that of T4 or T3, are/is applied topically to a target area in sufficient amount(s), local hypothyroidism is produced in this area. By utilizing this phenomenon, appreciable new therapeutic benefits can be achieved in proliferative dermatological conditions where an absolute or relative local excess of T4 (and T3) constitutes the etiological and/or disease-maintaining factor. This pertains not only to situations of systemic elevation of T4/T3 levels (thyrotoxicosis) but also to situations where a tissue area reacts locally to the hypermetabolic effect of T4/T3 (as in psoriasis, see below).

In consequence of the above, the present invention and the compounds defined in it are not aimed at achieving clinical and/or therapeutic effects in conditions characterized by reduced concentrations of T4/T3 in the target tissue. These include the dermal manifestations of hypothyroidism, where there is an absolute reduction in T4/T3, and also skin conditions where the tissue response to T4/T3 is reduced, i.e. where a relative deficiency of these hormones is the etiological factor.

The present invention is intended to be applied only in proliferative skin diseases, i.e. in conditions where the multiplication and/or growth of dermal cells is pathologically accelerated and/or their differentiation is deficient wholly or partly because of the sensitization of these cells to the effects of T4 and T3.

One example of such diseases is psoriasis. It manifests as scaly plaques on the skin, mostly on distal parts of the body such as elbows, knees, scalp and fingers. The lesions have an erythematous base covered by a thick layer of glossy, greasy, silvery grey scales. The psoriatic plaques vary in size, shape and number but they are always sharply demarcated from healthy skin. While the epidermal layer of healthy skin regenerates in about six weeks, cell production is elevated up to tenfold in psoriatic skin. Cells do not have the time to keratinise normally and, as a result, they are shed from psoriatic plaques in the form of characteristic scales.

The most practicable drug for topical treatment of mild or moderate psoriasis has until now been calcipotriol, a derivative of vitamin D. Its available pharmaceutical forms for topical therapy include ointments, creams and cutaneous solutions. The full effect of calcipotriol therapy is seen after 6 to 8 eight weeks of treatment. Still, complete removal of dermatitic plaques is achieved in only about 15% of cases (Guzzo C A, Lazarus G S, Werth V P (1996): Dermatological Pharmacology. In Goodman & Gilman's The Pharmacological Basis of Therapeutics, 9th ed. Eds. Hardman J G, Limbird L E, Molinoff P B, Rudden P W, Goodman Gilman A. McGraw-Hill, New York, p. 1593-1616).

When the present invention is utilized and topical pharmaceutical preparations containing the thyronine derivatives defined in the present invention are applied to psoriatic plaques, for instance, the therapeutic effect on the plaques is faster and more potent than even that produced by topical calcipotriol therapy.

Patent document WO 9640048, which bears a resemblance to the present invention, describes the topical use of thyroid hormone analogues in a group of proliferative and nonproliferative skin conditions. It is evident from the description and the associated exemplifying embodiments that the principle of said invention is not based on inhibiting or reducing the thyroid hormone effect of T4 and T3, i.e. actions characteristic of the present invention. That WO 9840048 is a distinct invention is indicated, firstly, by the fact that the effect of the invention is demonstrated by means of a dermal tissue model in which the pursued thyroid hormone activity is completely independent of the effects or presence of T4 or T3. Further, apart from the fact that a subsequent exemplifying embodiment of said invention mentions a beneficial effect on psoriasis from a topically administered thyroid hormone analogue (triiodothyroacetic acid), the same invention allows equally well T4 or T3 to be used for topical treatment of psoriasis. And yet these thyroid hormone analogues have an exclusively harmful topical effect on this disease. The present invention removes this fundamental disadvantage not only in practice but also logically. Thus, the invention described in WO 960048 and the present invention differ essentially from each other and are therefore distinct inventions.

The invention is characterized by what is stated in the patent claims.

### BRIEF DESCRIPTION OF THE DRAWINGS

FIGS. 1a-1f shows the topical effect of T4P, 3,3′,5,5′-tetraiodothyropropionic acid ointment on a previously untreated psoriatic plaque.

### DETAILED DESCRIPTION OF THE PRESENT INVENTION

Use of the invention will be described in the following exemplifying embodiment:

### Exemplifying Embodiment

Simultaneous topical treatment of four untreated, typically scaling and sharply demarcated dermatitic plaques (plaques 1-1) in a psoriatic patient was undertaken. Plaque 5 was left untreated for comparison.

The areas of the plaques at initiation of the treatment were as follows: plaque 1: 8.7 cm²; plaque 2: 8.1 cm²; plaque 3: 6.8 cm²; plaque 4: 6.1 cm²; plaque 5: 6.4 cm².

The following ointments were applied to plaques 1-4:

Plaque 1

Locobase.RTM. unctuous cream (Yamanouchi Europe B. V., Leiderup, Holland).

Plaque 2

Daivonex.RTM. calcipotriol ointment containing 50.mu.g calcipotriol per gram (L.o slashed.vens Kemiske Fabrik, Ballerup, Denmark).

Plaque 3

Locobase.RTM.D unctuous cream containing 500.mu.g 3,3′,5,5′-tetraiodothyroacetic acid, T4A, per gram (University Pharmacy, Helsinki, Finland).

Plaque 4

Locobase.RTM. unctuous cream containing 500 μg T4A per gram and 50 μL-thyroxine, T4, per gram (University Pharmacy, Helsinki, Finland).

Each ointment was applied to the plaques at 50 mg/cm² twice in 24 hours, corresponding to 5.mu.g calcipotriol per cm² per 24 hours for plaque 2, 50 μg T4A per cm² per 24 hours for plaques 3 and 4, and 5 μg T4 per cm² per 24 hours for plaque 4. Treatment was continued in this manner for 35 days.

The results are presented in Table 1 below, showing the change (reduction) in plaque area with time (days).

TABLE 1

| | 14 days | 21 days | 28 days | 35 days | |
|---|---|---|---|---|---|
| Plaque 1: | 0% | 0% | 0% | 0% | (Locobase .RTM.) |
| Plaque 2: | 35% | 55% | 65% | 85% | (Daivonex .RTM.) |
| Plaque 3: | 45% | 65% | 85% | 90% | (T4A) |
| Plaque 4: | 40% | 60% | 85% | 90% | (T4A + T4) |
| Plaque 5: | 0% | 0% | 0% | 0% | (no treatment) |

It can be seen from Table 1 that both the T4A ointment and the T4A+T4 ointment had a clear effect, and they were similar in therapeutic efficacy. Measured in terms of reduction in plaque area, their therapeutic efficacy was better than that of the calcipotriol ointment.

The therapeutic trial was continued from 35 days onward as follows:

Plaque 1

Locobase® ointment.

Plaque 2

No treatment.

Plaque 3

Locobase® ointment.

Plague 4

Locobase® ointment containing 50 μL-thyroxine, T4, per gram (University Pharmacy, Helsinki, Finland).

Plaque 5

No treatment.

As previously, the ointments were applied to the target plaques at 50 mg/cm² twice in 24 hours. Thus, the dose of T4 to plaque 4 was 5 μg T4 per cm² per 24 hours.

The results are presented in Table 2 below.

TABLE 2

| | 42 days | 50 days | 62 days | 70 days | |
|---|---|---|---|---|---|
| Plaque 1: | 0% | 0% | 0% | 0% | (Locobase .RTM.) |
| Plaque 2: | 85% | 80% | 80% | 70% | (no treatment) |
| Plaque 3: | 95% | 95% | 90% | 80% | (Locobase .RTM.) |
| Plaque 4: | 70% | 25% | -10% | −50% | (T4) |
| Plaque 5: | 0% | 0% | 0% | 0% | (no treatment) |

It can be seen from Table 2 that although some rash did return over four weeks to those skin lesions on which the treatment with calcipotriol or T4A ointments had been discontinued, the T4 ointment in fact reinstated the rash completely to plaque 4 previously treated with T4A+T4 ointment. Indeed, the T4 ointment increased the area of plaque 4 by 50% compared with baseline (Table 1). Therefore, it can be concluded that psoriatic rash is worsened by topically applied T4 (T3) and that T4A completely inhibits this effect and is also therapeutically efficacious.

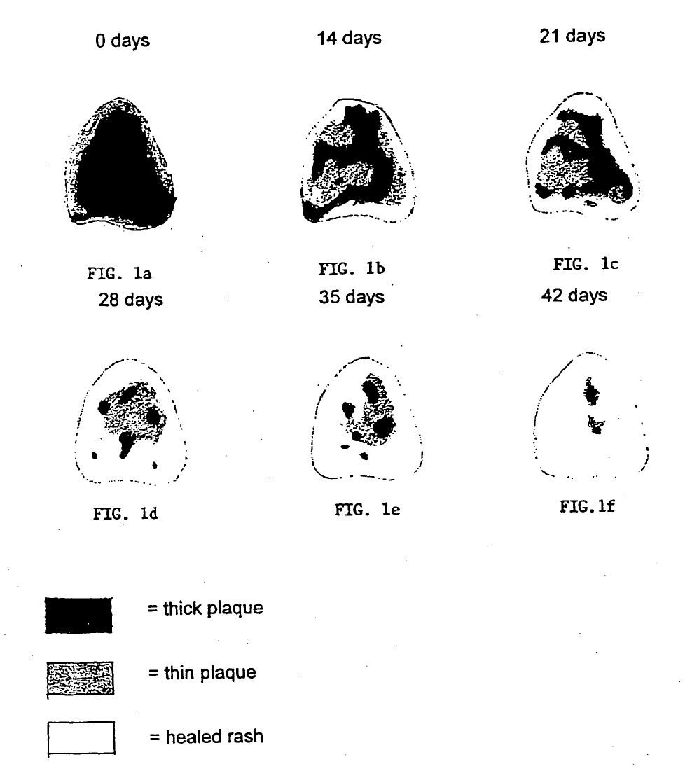

The topical effect of T4P, 3,3′,5,5′-tetraiodothyropropionic acid ointment on a previously untreated psoriatic plaque, on psoriasis was studied in a therapeutic design identical with the above. The dosage of T4P was 25 μg/cm² twice per 24 hours for 35 days. The follow-up period was 42 days. The ointment base was the same at the same concentration and dosage as T4A previously, T4P was found to completely inhibit the effects of T4 and to be equal to T4A in therapeutic efficacy (FIGS. 1a-1f). Effect of 3,3′,5,5′-tetraiodothyropropionic acid (T4P) ointment on a previously untreated psoriatic plaque. The dosage of T4P was 25 μg/cm² twice per 24 hours for 35 days. The follow-up period was 42 days.

The target effect of the thyroid hormone compounds 3,3,5,5′-tetraiodothyroacetic acid (T4A) and 3,3′,5,5′-tetraiodothyropropionic acid (T4P) used topically as described above is the counteraction of thyroid hormone compounds on the proliferative changes of the skin, resulting in a further effect in psoriatic skin changes, called in turn antipsoriatic effect, not necessarily being identical with antiproliferative effect. If the former effect is prominent enough, like it becomes in the case of continuous topical application of T4A and T4P, the result is an improvement of psoriatic rash as it is shown above.

WO 9640048 introduces in addition to several other thyroid hormone compounds L-thyroxine (T4), L-triiodothyronine (T3), D-thyroxine (D-T4), 3,5,3′-triiodothyroacetic acid (T3A), but equally also T4A and T4P for the treatment of psoriasis, making no difference between proliferative or antiproliferative effects of these or any other thyroid hormone compounds. WO 9640048 simply teaches that all these compounds possess an antipsoriatic effect. In other words according to this invention all the concerned compounds are suitable for the topical treatment of psoriasis.

WO 9640048 does not, however, give any appropriate guidelines in order to distinguish with the clinical antipsoriatic efficacy between the various thyroid hormone compounds let alone their clinical therapeutic or possible adverse effects. In WO 01/76589 it has already been shown that despite its antipsoriatic property, as defined above, topically administered T4 clinically worsens psoriatic rash, which is an adverse effect over antipsoriatic effect. In WO 01/76589 this adverse effect of T4 could be reversed by applying T4A. Following verification of this capability of T4A of reducing adverse effects of topically applied T4 there has emerged an open novel question: Would T4A be additionally a compound suitable for an improvement of the antipsoriatic efficacy of other thyroid hormone compounds?

This was studied successfully by the following clinical experiments:

### Additional Exemplifying Embodiment

Simultaneous topical treatment of four untreated, typically scaling and demarcated dermatitic plaques (plaques 1-4) in a psoriatic patient was undertaken. Plaque 5 was left untreated for comparison.

The areas of the plaques at initiation of the treatment were as follows: plaque 1: 5.3 cm², plaque 2: 4.9 cm², plaque 3: 6.4 cm², plaque 4: 5.3 cm²; plaque 5: 5.6 cm².

The following ointments were applied to plaques 1-4:

Plaque 1

Triacana® cream containing 200 μg 3,5,3′-triiodothyroacetic acid, T3A, (triac, tiratricol) per gram.

Triacana® cream is a commercial medicinal preparation having been available in France since 1976. (The product information regarding the cream is set forth in the entry of ‘Triacana crème’ on editorial pages and pages 1774-1775 in the French dictionary of pharmaceuticals: ‘Dictionnaire Vidal®’ Paris, O.V.P., 1990, the disclosure of which is incorporated herein by reference.)

Plaque 2

Triacana® cream plus Locobase® unctuous cream containing 500 μg 3,3′,5,5′-tetraiodothyroacetic acid, T4A, per gram (University Pharmacy, Helsinki, Finland. Ex tempore-preparation).

Plaque 3

Locobase® unctuous cream containing 2500 μg D-thyroxine, D-T4, per gram (University Pharmacy, Helsinki, Finland. Ex tempore-preparation).

Plaque 4

Locobase® unctuous cream containing 2500 μg D-thyroxine, D-T4, per gram (University Pharmacy, Helsinki, Finland. Ex tempore-preparation) plus Locobase® unctuous cream containing 500 μg 3,3′,5,5′-tetraiodothyroacetic acid, T4A, per gram (University Pharmacy, Helsinki, Finland. Ex tempore-preparation).

Triacana® cream was applied to plaques 1 and 2 at 125 mg/cm² twice in 24 hours, corresponding to 50 μg 3,5,3′-triiodothyroacetic acid per cm² per 24 hours.

T4A-ointment was applied to plaque 2 concurrently with Triacana® cream at 50 mg/cm² twice in 24 hours, corresponding to 50 μg 3,3′,5,5′-tetraiodothyroacetic acid per cm² per 24 hours.

D-T4-ointment was applied to plaques 3 and 4 at 50 mg/cm² twice in 24 hours, corresponding to 250 μg D-T4, per cm² per 24 hours.

T4A-ointment was applied to plaque 4 concurrently with the D-T4-ointment at 50 mg/cm² twice in 24 hours corresponding to 50 μg/cm² 3,3′,5,5′-tetraiodothyroacetic acid per cm^{2 per} 24 hours.

Treatment was continued in this manner for 35 days.

Results:

Plaque 1: During the whole treatment period no visible changes took place in the area or the appearance of the plaque.

Plaque 2: At the end of the treatment period, the area of the plaque was diminished by 90%.

Plaque 3: During the whole treatment period no visible changes took place in the area or appearance of the plaque.

Plaque 4: At the end of the treatment period, the area of the plaque was diminished by 95%.

Plaque 5: No changes.

### CONCLUSIONS

Topically administered T3A and D-T4 did not have any visible effect on psoriatic rash in these experiments.

On the contrary, topically administered T4A had a separate and prominent antipsoriatic effect on psoriatic rash despite the presence of other thyroid hormones, at least T4, T3, T3A and D-T4. The latter hormones have an antipsoriatic effect according to the teachings of WO 9640048 and the corresponding U.S. Pat. No. 6,221,911.

Thus, the effect of T4A was unexpected and surprising and involves an inventive step.

1. A method for treating a proliferative skin pathology caused by 3,3′,5,5′-tetraiodo-L-thyronine (T4), which comprises administering a therapeutically effective topical amount of 3,3′,5,5′-tetraiadothyroacetic acid (T4A) or 3,3′,5,5′-tetraiodothyropropionic acid (T4P), to the skin of a patient in need thereof.

2. The method of claim 19, wherein origin of T4 is endogenous.

3. A method for treating a proliferative skin pathology caused by 3,3′,5,5′-tetraiodo-L-thyronine (T4), which comprises administering a therapeutically effective topical amount of 3,3′,5,5′-tetraiadothyroacetic acid (T4A) or 3,3′,5,5′-tetraiodothyropropionic acid (T4P), to the skin of a patient in need thereof, wherein origin of T4 is its topical administration.

4. The method of claim 19, wherein the therapeutic effective amount is about a daily amount of about 0.05 mg of T4A or T4P per cm² of the diseased skin.

5. The method of claim 20, wherein the therapeutic effective amount is about a daily amount of about 0.05 mg of T4A or T4P per cm² of the diseased skin.

6. The method of claim 21, wherein the therapeutic effective amount is about a daily amount of about 0.05 mg of T4A or T4P per cm² of the diseased skin.

7. The method of claim 19, wherein the administration of T4A, T4P, or both is either concurrent with topical T4 or precedes or succeeds T4.

8. The method of claim 20, wherein the administration of T4A, T4P, or both is either concurrent with topical T4 or precedes or succeeds T4.

9. The method of claim 21, wherein the administration of T4A, T4P, or both is either concurrent with topical T4 or precedes or succeeds T4.

10. The method of claim 22, wherein the administration of T4A, T4P, or both is either concurrent with topical T4 or precedes or succeeds T4.

11. A method for enhancement of the antipsoriatic effect of thyroid hormones in treatment of psoriasis comprising topically administering a therapeutically effective amount of 3,3′,5,5′-tetraiadothyroacetic acid (T4A) to the skin of a patient in need thereof, wherein the thyroid hormones is 3,5,3′-triiodothyroacetic acid, D-thyroxine (D-T4) or L-thyroxine (T4) and L-triiodothyronine (T3).
